(19) United States
(12) Patent Application Publication
(10) Pub. No.: US 2015/0297095 A1
(43) Pub. Date: Oct. 22, 2015

(54) REVERSIBLE THERMOCHROMATIC LIQUID CRYSTAL CONTACT MEANS FOR SELF-DETECTION OF SUBDERMAL ABNORMAL CELL METABOLISM

(71) Applicant: Fried Robert, New York, NY (US)

(72) Inventor: Fried Robert, New York, NY (US)

(21) Appl. No.: 14/691,388

(22) Filed: Apr. 20, 2015

Related U.S. Application Data
(60) Provisional application No. 61/981,990, filed on Apr. 21, 2014.

(51) Int. Cl. A61B 5/01; A61B 5/00

(52) U.S. Cl. CPC A61B 5/015; A61B 5/742; A61B 5/4312

(57) ABSTRACT

A device for routine self-examination and self-detection of suspected human subcutaneous lesions and possible cancerous tumors, by “hot spot” observation of color change on a screen that is treated with thermochromatic reversible “liquid crystal” materials. In embodiments, elevated metabolic activity causing elevated abnormal tissue temperature is reflected on the screen of the device by color differences. In embodiments, the device may be portable, handheld, inexpensive, and easy-to-use to encourage widespread adoption and use.

### PRIORITY CLAIM

This application claims priority to U.S. Provisional Patent Application No. 61/981,990, filed Apr. 21, 2014 and titled, “REVERSIBLE THERMOCHROMATIC LIQUID CRYSTAL CONTACT MEANS FOR SELF-DETECTION OF SUBDERMAL ABNORMAL CELL METABOLISM,” the contents of which is incorporated by reference in its entirety.

### BACKGROUND

The present invention relates to a portable, reversible chromatographic device that will indicate normal and abnormally elevated subdermal cell metabolic activity and consequent higher temperature by signature color differences on contact with the skin. Identifying a “hot spot” on the screen alerts the user to seek immediate medical attention.

In an example, abnormal subdermal metabolic activity may be caused by cancer of the breast, which is the most common cancer in women. According to the American Cancer Society, the chance of a woman having invasive breast cancer during her life is about 1 in 8 and the chance of dying form breast cancer is about 1 in 36.

In just 2014 it is expected that 232,670 new cases of invasive breast cancer will be diagnosed in women, and 40,000 will die from the disease. Over 60,000 new cases of carcinoma in situ—the earliest form of breast cancer—will be detected.

Early detection of breast cancer is critical, and considered to be one of the best predictors of successful treatment. According to the California Breast Cancer Research Program, a recent study from the M.D. Anderson Cancer Center (University of California) that compared length of survival of metastatic breast cancer patients treated at their institution in five-year increments, found that median survival doubled to 51 months (range 33-69 months) in 1995-2000 from a median of 27 months (range 21-33 months) only five years earlier, 1990-1994. Five years after their diagnosis with metastatic disease, 40 percent of these patients were still alive, as compared to 29 percent during 1990-1994. At the initiation of their study, during the period 1974-1979, only 10 percent of patients were still alive at the five-year mark, and the median survival was only 15 months (range 11-19 months) (Giordano, Buzdar, Kau, et al., 2002; http://cbcrp.org/publications/papers/mayer/page_{—}03.php). While great strides have been made in detection and treatment, there is tremendous room for improvement.

Breast cancer is by far the most frequent cancer among women with an estimated 1.38 million new cancer cases diagnosed worldwide in 2008 (23% of all cancers). It is now the most common cancer both in developed and developing regions with 690,000 new cases estimated in each region (population ratio 1:4). Incidence rates vary from 19.3 per 100,000 women in Eastern Africa to 89.9 per 100,000 women in Western Europe, and are high (greater than 80 per 100,000) in developed regions of the world (except Japan) and low (less than 40 per 100,000) in most of the developing regions (Ferlay, Shin, Bray, et al., 2008).

From 2001 to 2010 the overall incidence of breast cancer in women has remained level, without statistically appreciable improvement. Deaths across almost every ethnic class were down over that time period, with the exception of black women, where there was an increase of 0.5% per year.

Men are not exempt from the reach of breast cancer with 2,039 men being diagnosed in 2010 and 439 succumbing to the disease.

Screening is effective in reducing deaths from breast cancer in women. There are two prominent current conventional medical breast cancer screening procedures, namely, mammography and clinical thermography.

Mammography uses low-energy X-rays as a diagnostic tool to detect breast cancer by signature masses and/or evident microcalcifications. Clinical thermography typically relies on infrared optical imaging methods to detect tumors in subcutaneous tissues by differentiating their characteristically higher metabolic temperatures than those in surrounding healthy tissue.

The World Health Organization guidelines suggest a mammogram every two years, with routine self-examination in-between. However, there are drawbacks.

Mammographic examination is recommended on a biennial cycle in part to reduce a patient's exposure to the X-ray radiation used in the examination and which can be harmful in cumulative doses. Economic considerations certainly play a role as well.

However, a two-year interval between examinations is thought to be too great and health authorities concerned with breast cancer in women have therefore recommended and taught methods of breast self-examination by palpation. It should be noted that breast self-examination by palpation—a form of examination that was once recommended in the gap between mammograms—is reported by the American Cancer Society (ACS) and the World Health Organization (WHO) to be ineffective.

Studies have shown that self-examination by palpation does not actually improve an individual's chance of surviving an incidence of breast cancer. Indeed, regular self-examination by palpation greatly increases the likelihood that a needless biopsy will be taken of a benign breast mass. See, e.g., http://www.webmd.com/breast-cancer/news/20080715/breast-self-exam-no-survival-benefit. These biopsies can be costly, as well as physically and emotionally disruptive to the patient and his or her family.

Clinical infrared optic scanning and recording apparatus used in conventional breast diagnostic thermography is an alternative.

It has been shown that optical scanning thermography is an effective method of breast tumor detection. While clinical infrared optical scanning thermography is safer than X-ray mammography, frequent scanning is neither practical nor is it affordable.

Reversible liquid crystal contact thermography has also been proven scientifically to be as effective as clinical infrared optical scanning thermography in detecting abnormal cell activity in breast subdermal tissue by corresponding color changes on a reversible liquid crystals screen reflecting that signature of underlying elevated metabolic heat production.

However, there currently exists no means of reversible liquid crystal contact chromatography in a form suitable to breast self examination.

Accordingly, given the vast number of patients impacted by the disease, there is a significant need for a new strategy for ad-lib self-examination that bridges the gap between clinical examinations allowing for the possibility of early detection between exams.

There is a further need for a screening device that is reliable, portable, inexpensive to mass produce, and easy to use.

There is a further need for a screening device that permits do-it-at-home use where the need is greatest between clinical examinations, or screening use in geographic locations of the world where clinical examinations are too costly or for other reasons unavailable.

### SUMMARY OF THE INVENTION

In embodiments, a screening tool for use in detecting subcutaneous thermal abnormalities comprising is disclosed. An elongate handle portion may be provided with a frame portion connected to said handle portion, and a thermochromatic screen supported by said frame portion. In embodiments, the thermochromatic screen is formed from an opaque backing material impregnated on at least one side by a plurality of thermal-sensitive liquid crystals, and the thermal-sensitive crystals are responsive to a predetermined temperature range for the desired application.

In embodiments, the elongate handle is formed from a material that is heat resistant such that the amount of heat transmitted from an operator's hand to said thermochromatic screen is minimized, and may be formed from plastic, polyvinyl chloride, polypropylene, wood, ceramic, cardboard, or rubber. In embodiments, the frame portion and elongate handle portion are integrally formed from a single piece of material. In further embodiments, the frame portion and said elongate handle portion are permanently joined during manufacture. The thermochromatic screen may be joined to the frame portion such that said thermochromatic screen can be applied flush to a surface. The frame portion may also comprise two discrete sections that are joined during manufacture and further wherein a portion of said thermochromatic screen is held between said discrete sections.

In alternate embodiments, a screening tool is disclosed in which a plurality of opposing handle portions are provided. A thermochromatic screen formed from an opaque backing material impregnated on at least one side by a plurality of thermal-sensitive liquid crystals may be substantially supported by said handle portions In embodiments, the thermal-sensitive crystals are responsive to a predetermined temperature range for the desired application.

In further embodiments, a method of using the device of the present invention is disclosed, the steps including providing a detector having a thermochromatic screen formed from an opaque backing material impregnated on at least one side by a plurality of thermal-sensitive liquid crystals, wherein the thermal-sensitive crystals are responsive to a predetermined temperature range for the desired application. The detector may then be applied to the surface of the region to be tested. The operator may then wait for the thermochromatic screen to register and examine the thermochromatic screen for a evidence of a color gradient.

### BRIEF DESCRIPTION OF THE DRAWINGS

Throughout the course of the following detailed description, reference will be made to the drawings in which like reference numbers identify like parts and in which:

FIG. 1 is a view of an embodiment of the invention;

FIG. 2 is a view of an alternate embodiment of the invention;

FIG. 3 illustrates how an embodiment of the present invention may be used by a consumer for self-detection, or by a health care worker for screening; and

FIG. 4 illustrates how an alternative embodiment of the present invention may be used by a consumer for self-detection, or by a health care worker for screening.

### DETAILED DESCRIPTION OF THE PREFERRED EMBODIMENT

Detailed embodiments of the disclosure are disclosed herein; however, it is to be understood that the disclosed embodiments are merely exemplary and may be embodied in various forms. Therefore, specific details disclosed herein are not to be interpreted as limiting, but merely as a basis for the claims and as a representative basis for teaching one skilled in the art to variously employ the disclosure in virtually any appropriate manner, including employing various features disclosed herein in combinations that might not be explicitly disclosed herein.

Further, while the disclosure utilizes breast cancer as an illustrative medical condition, the present invention may be used with any condition where surface temperature irregularities may be an indicator of a surface or a subsurface medical condition, or where it is otherwise desirable or useful to identify such thermal inconsistencies.

In embodiments, a handheld screening tool or detector for use in detecting regions of elevated temperature across the body is disclosed. In embodiments, the detector is used for self-detection of suspected subcutaneous lesions and possible cancerous tumors, by “hot spot” observation of color change on a display screen of the device. These “hot spots” may be caused by subcutaneous abnormally elevated metabolic activity, which may cause elevated abnormal surface tissue temperature, and indicate that medical attention is warranted.

In embodiments, the device may be applied to the surface of the skin so that a few seconds of contact over any region will show any existing abnormal subdermal cell metabolic heat by color contrast with surrounding regions. Lifting the screen from the skin may allow the screen to return to its pre-exposure color in a few seconds and then the process can be repeated with another region.

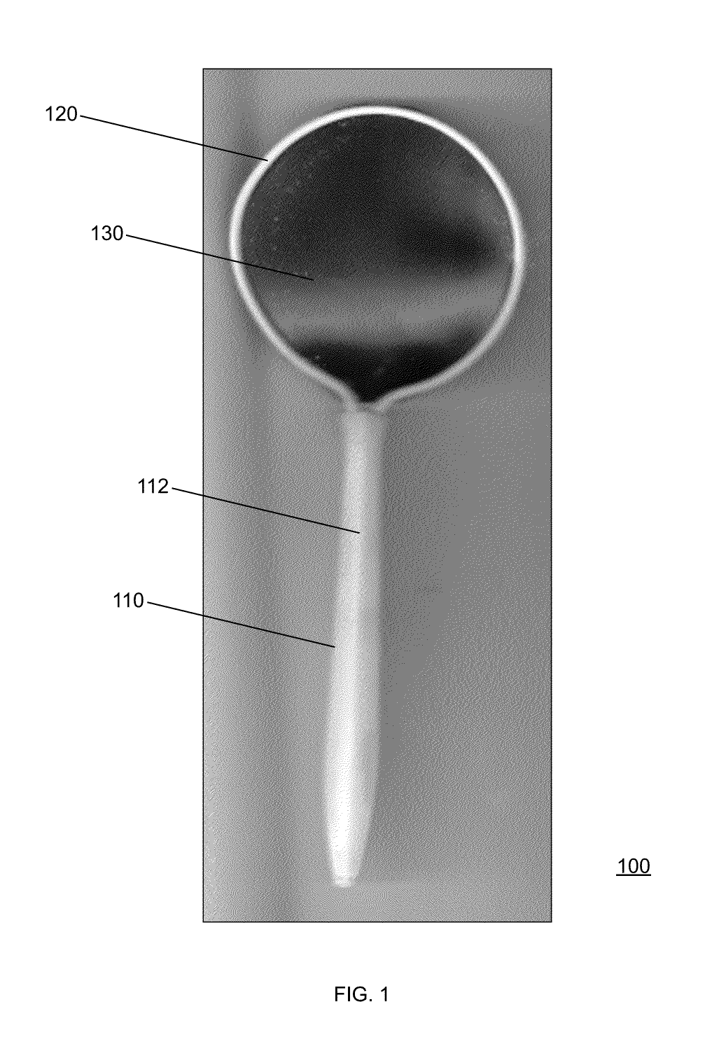

Referring to FIG. 1, an embodiment of the detector of the present invention is disclosed. A detector device 100 comprises a handle portion 110, frame portion 120, and thermochromatic screen 130.

In embodiments, handle portion 110 may be ergonomically shaped to accommodate the hand of a user of the device. In embodiments, handle 110 is substantially cylindrical in shape with a distal end that tapers for safety and comfort.

In embodiments, handle 110 may comprise design elements that enhance the aesthetic appeal or utility of the device. For example, any combination of tacky material for gripping the device, textured elements such as grooves or ridges, graphics, or text could be incorporated into handle 110. In embodiments, handle 110 may comprise structural elements that enhance the resilience or sturdiness of the device. For example, where a relatively soft and lightweight material such as aluminum is used, handle 110 may comprise a ridge 112 that runs along handle 110. Such a ridge 112 can improve the stability and durability of the device and allow it to be manufactured from a less costly and lighter material.

In embodiments, handle portion 110 may be formed from any material, or combination of materials, and in any manner, that will resist transmitting excess heat from the operator's hand to frame portion 120 and thermochromatic screen 130, which could cause an inaccurate reading. In embodiments, the material used for handle 110 may be lightweight and/or inexpensive, which can make the device easier to use and available to a wider audience.

For example, normal human skin temperature is approximately 91.0° F. at a neutral room temperature of 72° F., and abnormal subdermal cell metabolic activity presents as a temperature rise of 1-3° F. In embodiments, handle portion should not appreciably raise the temperature of thermochromatic screen 130 during a scan of average duration, as discussed below, such that it causes a false reading.

In embodiments, it has been discovered that suitable materials for the handle portion 110 that meet the criteria of thermal resistance, weight, and cost include, without limitation, plastics such as polyvinylchloride or polypropylene, wood, ceramic, cardboard, rubber, or similar materials. Other materials meeting any one or more of the criteria of thermal resistance, weight, and cost may be used and still keep within the scope of the present invention.

In embodiments, a frame portion 120 may be formed at an end of handle 110 and support a portion of thermochromatic screen 130.

In embodiments, handle portion 110 and frame portion 120 are discrete components that are permanently joined during manufacture such as by an adhesive, epoxy, welding, brazing, riveting, or the like. In further embodiments, handle portion 110 and frame portion 120 may be integrally formed, such as from a single piece of material. In further embodiments, frame portion 120 comprises two or more sections that may be fastened together before being joined to handle portion 110 by any of the foregoing methods.

In embodiments, frame portion 120 may hold thermochromatic screen 130 in position so that thermochromatic screen 130 may be placed atop an operator's skin. As discussed below, the thermally sensitive thermochromatic screen 130 should be isolated from the operator's hand for the most accurate reading. In embodiments, frame portion 120, in conjunction with handle potion 110, may isolate thermochromatic screen 130 from the operator's hand, and also hold the thermochromatic screen in position during operation. Accordingly, in embodiments, frame portion 120 may hold thermochromatic screen 130 in a substantially planar configuration.

In embodiments, frame portion 120 may be formed from a length of wire that has been sized and shaped to accommodate the size and shape of thermochromatic screen. In embodiments, thermochromatic screen 130 may be joined to frame portion 120 by means of an adhesive, epoxy, or any other method that is durable and does not interfere with the contact between thermochromatic screen 130 and the user's skin.

In other embodiments, frame portion 120 may comprise two or more pieces that are joined together with a portion of thermochromatic screen positioned and held therebetween. One such example is interlocking molded plastic components that snap fit with thermochromatic screen 130 between the halves. Myriad other means for framing and stabilizing thermochromatic screen 130 may be used and stay within the scope of the current invention.

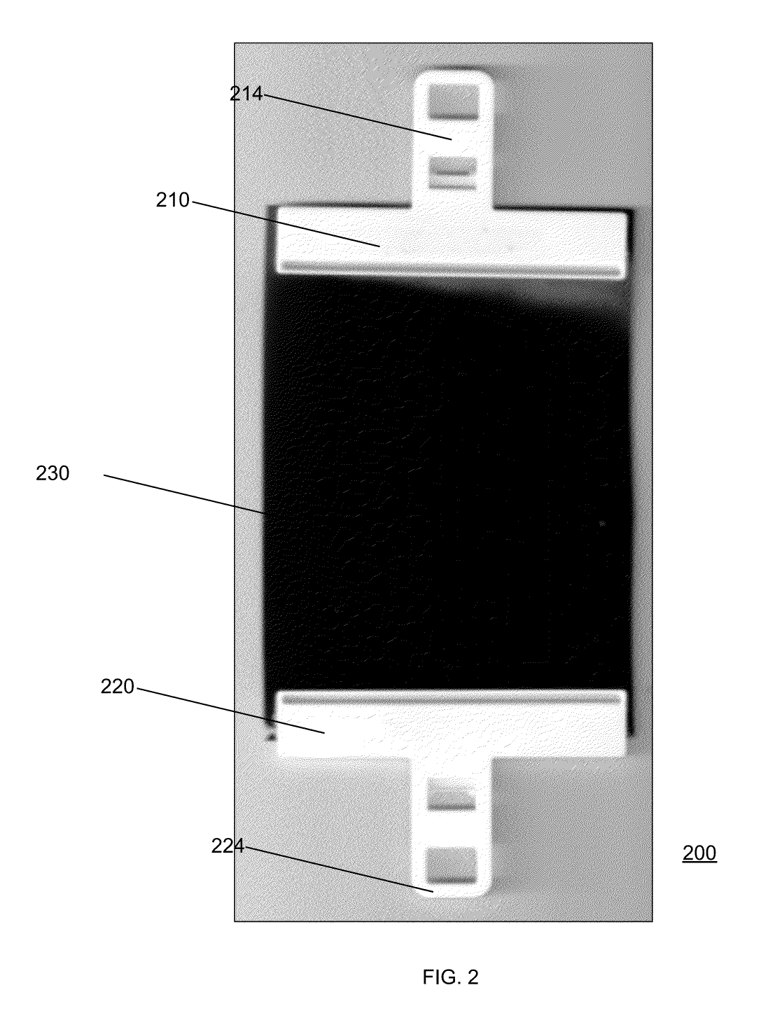

Referring to FIG. 2, an alternative embodiment of the detector 200 of the present invention is shown. Referring to FIG. 2, detector 200 may comprise handle portions 210-220 and thermochromatic screen 230.

In embodiments, handle portions 210-220 may comprise a length of substantially rigid material that can hold thermochromatic screen 230 in position along one end of the screen. In embodiments any joining method described in connection with the embodiment of FIG. 1 may be used with the embodiment of FIG. 2. For example, in embodiments, thermochromatic screen 230 is joined to handle portions 210-220 by an adhesive, epoxy, or any other means that provides durability and stability.

In embodiments, handle portions 210-220 may each be joined to an edge of thermochromatic screen 230. In the configuration shown in FIG. 2 handle portions 210-220 may also perform the function of the frame portion of other embodiments. By integrating the frame into the handle portions 210-220, the detector may be simpler to manufacture and less costly.

When supported on only two sides as shown in FIG. 2, the flexibility of thermochromatic screen 230 is enhanced and the device may thus be applied to a portion of the body that is curved. This may be particularly useful for inspecting a breast or other curved area.

In embodiments, handle portions 210-220 may be symmetrical and attach to opposing edges of thermochromatic screen 230.

Handle portions 210-220 may also comprise design elements that enhance the aesthetic appeal or utility of the device including, for example, any combination of tacky material for gripping the device, textured elements such as grooves or ridges, graphics, or text could be incorporated into handle portions 210-220. In embodiments, handle portions 210-220 may comprise structural elements that enhance the resilience or sturdiness of the device, which may allow it to be manufactured from a less costly and lighter material.

Handle portions 210-220 may be formed from any material, or combination of materials, and in any manner, that will resist transmitting heat from the operator's hand to thermochromatic screen 230. Further, in embodiments, the material used for handle portions 210-220 may be lightweight and/or inexpensive, which can make the device easier to use and available to a wider audience.

The materials suitable for the embodiment shown in FIG. 2 are similar to those for the embodiment of FIG. 1 and include, for example, plastics such as polyvinylchloride or polypropylene, wood, ceramic, cardboard, or rubber. Other materials meeting any one or more of the criteria of thermal conduction/transmission resistance, weight, and cost may be used and keep within the scope of the present invention.

In embodiments, extensions 214-224 may extend from one or both handle portions 210-212 to provide additional or more ergonomic surface area for the user to grip, or to provide additional thermal resistance by positioning the user's hand further away from thermochromatic screen 230 when in use.

In embodiments, handle portions 210-220 further comprise handle protrusions 214 and 224 that permit the user to grasp the device with the fingertips. In embodiments, handle protrusions 214-224 are sized and shaped to accommodate the fingers of a person of average size or target market. In embodiments, handle protrusions 214-224 may further comprise structural elements that enhance the resilience or sturdiness of the device such as, for example, lateral ridges.

Thermochromatic Screen

It has been found that thermochromatic liquid crystals register and respond to a local temperature change as a local color change. The color of these liquid crystals can thus be used within reasonable limits as an accurate indicator of temperature.

As the temperature of a thermochromatic liquid crystal changes, the wavelength of light reflected by the crystal also changes so that changes in crystal temperature will present as changes in color. Different types of liquid crystals are responsive to different wavelengths of light. In embodiments, the liquid crystals are dispersed within a polymer matrix that provides protection to the crystals.

In embodiments, thermochromatic screen 130 (or 230) may be formed from a material comprising thermal-sensitive liquid crystals, or any other material that is lightweight, inexpensive, and registers a visual representation of a temperature gradient.

Unless otherwise noted, the discussion of thermochromatic screen 130 in this section applies equally to thermochromatic screen 230, or any other thermochromatic screen of the present invention.

In embodiments, thermochromatic screen 130 (or 230) may comprise a biaxially-oriented polyethylene terephthalate (e.g., Mylar™) sheet that may be dark on one side and impregnated or coated with liquid crystals on the other side. The dark side absorbs any light that would otherwise be transmitted through the liquid crystal, allowing the user to view the temperature gradient—through the sheet—without interference. In embodiments, liquid crystals may be coated onto any material that does not interfere with the user's ability to view the color gradient on thermochromatic screen 130 (or 230).

In embodiments where the crystals are dispersed in or on a protective coating, the crystals will be resistant to contamination by dust, dirt, or any other contaminant that could cause the crystals to lose their thermochromatic properties.

In embodiments, thermochromatic screen 130 (or 230) is formed by taking a large, commercially available sheet of material and cutting individual portions to reduce cost. For example, a commercially available 12″×12″ sheet could be cut to nine separate 4″×4″ sheets, which would be significantly less expensive than sourcing individually manufactured 4″×4″ sheets.

In embodiments, the thermochromatic liquid crystals selected for thermochromatic screen 130 (or 230) are responsive to a specific temperature range. For example, in the case of measuring the temperature of the human body, it has been found that abnormal subdermal cell metabolic activity presents as a temperature rise of 1-3° F. In embodiments, the liquid crystal material chosen should be able to register a temperature increase of that range over the average human skin temperature of 91.0° F. at room temperature. In embodiments, the liquid crystal material chosen may have a wider sensitivity to account for variations in individual body temperatures. In a preferred embodiment, the liquid crystal material chosen is sensitive in the range of 86° F. to 95° F. Other applications may require that the temperature range or sensitivity be modified.

It should be noted that these ranges are merely a guidelines and that any number of temperature ranges and thermochromatic sensitivities may be used with the present invention.

In embodiments, liquid crystals are chosen according to the amount of time that the crystals take to register a temperature as a change in reflective wavelength and thus the color gradient appearing to the user. If the response time is too slow, a temperature change may not register on the device as the operator performs a scan. If the response time is overly rapid, the cost of manufacturing or materials may be needlessly high. In embodiments, the thermochromatic screen responds to temperature changes within the range of 86° F. to 95° F.

In embodiments, the thermochromatic screen 130 (or 230) may be a single portion that is rectangular, square, round or any other polygonal shape. In embodiments, thermochromatic screen is sized according to the intended use. For example, in the case of scanning a human breast, thermochromatic screen 130 (or 230) may be large enough so that the user is able to cover and scan a sufficient area with each reading without needlessly prolonging the scan, which could discourage use. In the same example, thermochromatic screen 130 (or 230) may also be small enough such that areas that are difficult to scan (e.g., curved areas, hard to reach areas) may be examined. Configurations of 3″×4″, 3″×3″, and a 2.5″ diameter circle have all been found to be suitable to carry out the invention, though any size and configuration that meet the foregoing criteria remain within the scope of the present invention.

In embodiments, detector 100 may be packaged or distributed with thermochromatic screens of varying sizes and shapes to accommodate different scanning areas and body types. Detector 100 may also be sold or distributed with a single size of thermochromatic screen for a particular scanning area or body type, as part of a wider product lineup that includes thermochromatic screens of varying shapes and sizes.

In embodiments, thermochromatic screen 130 (or 230) may be durable. In embodiments, a Mylar or polymer backing is preferred to insure such durability. In embodiments, thermochromatic sheet 130 may be formed from a material such as Temperature Sensitive Liquid Crystal Sheets (R500009 1665-13347) available from Edmund Optics of Barrington, N.J., although any material exhibiting the foregoing properties would fulfill the requirements of the present invention.

Other than the shape and size of the material used, the thermochromatic screen 130 is substantially identical to thermochromatic screen 230. The discussion herein concerning thermochromatic screen 130 equally applies to thermochromatic screen 230.

### EXAMPLES

Three exemplary embodiments of the present invention have been developed. The following embodiments are meant only to be exemplary for the purpose of better understanding the present invention. Myriad embodiments are contemplated as coming within the scope of the present invention.

### Example 1

A thermochromatic liquid crystal polyester screen fastened between two wood, plastic, rubber, sturdy cardboard or other heat insulators “handles,” allows the user to hold it over the breast or other skin surface without direct hand contact of the patch, or strip, thus preventing hand temperature from altering the liquid crystal basal color. The strip is held over an area until the color stabilizes. The user may then apply the same procedure to the other breast.

### Example 2

A thermochromatic liquid crystal polyester screen in the general shape of a female human brassiere cup with two wood, plastic, sturdy cardboard or other insulators “handles,” one at each side, allows the user to hold it over the breast skin surface without direct hand contact thus preventing hand temperature from altering the liquid crystal basal color. The screen is held over a breast until the color stabilizes. The user may then apply the same procedure to the other breast.

### Example 3

A thermochromatic liquid crystal polyester sheet or film circular, square or rectangular patch, smaller than the strip or sleeve, fastened between to a wood, plastic, sturdy cardboard or other heat insulators “handle,” allows the user to hold it over the breast or other skin surface without direct hand contact of the patch, or strip, thus preventing hand temperature from altering the liquid crystal basal color. The strip can be moved to and held over any smaller skin surface area until the color stabilizes. The user may then apply the same procedure to the other skin surface areas.

### USE OF THE DEVICE

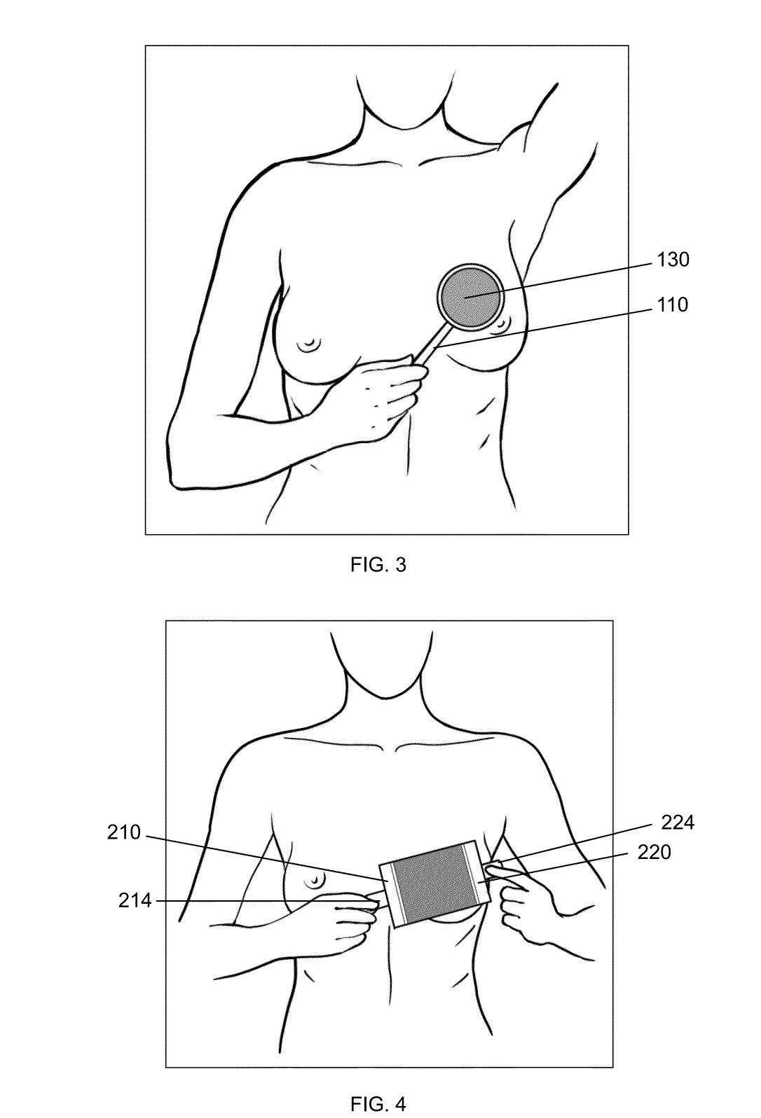

Referring to FIG. 3, a method of using the detector 100 of FIG. 1 is shown.

In embodiments, to perform a scan using the device, a user may grip the device on handle portion 110 and apply thermochromatic screen 130 against the surface of the area to be tested. In embodiments, the entirety of thermochromatic screen 130 may be placed flush against the surface of the skin.

In embodiments, the operator may then wait for the thermochromatic screen 130 to register. Registration is the process of the liquid crystals in the thermochromatic screen 130 reacting to the temperature and registering that temperature as a color.

The amount of time that it takes for thermochromatic screen 130 to register will depend on the characteristics of the particular material used for thermochromatic screen 130 and the user may be informed of this registration time in advance of using the device.

After the operator has waited for the prescribed registration time, the operator may look at thermochromatic screen 130 for evidence of a color gradient.

In embodiments, the liquid crystal material used in the thermochromatic screen will register different temperatures within a range as different colors.

In embodiments, the thermochromic screen may register consistent skin temperature as substantially black, while areas higher temperature register as a lighter color. Such a reading indicates a thermal anomaly and that urgent medical consultation may be warranted. A color that is consistent in shade across the surface of thermochromatic screen indicates a lack of thermal anomaly and that medical attention may not be warranted.

Continuing the use of the device, in embodiment, the user lifts the device from the skin and waits for thermochromatic screen 130 to return to a baseline room temperature. The particular temperature and length of time will depend on the materials used and should be communicated to the user in advance of the scan.

After the prescribed waiting period, the screen is then moved to a different location and the procedure is repeated until all areas of the surface of the breasts or body have been observed.

In embodiment, there is no known harm from exposure of skin to the detectors nor frequent use of these detectors, which encourages their use and possibly early detection of disease.

Referring to FIG. 4, the method of use of the embodiment of FIG. 2 is shown. The method of using the FIG. 2 embodiment is substantially similar to that of FIG. 1 with the exception of handles 210 and 220.

In use, the operator grasps each of handle portions 210 and 220 (at protrusions 214-224 if present). The operator may pull handle portions 210 and 220 in opposing direction to pull thermochromatic screen 230 taut against the skin.

In embodiments, the user then proceeds as described above, waiting for the registration period and repeating as necessary across the area to be tested.

In alternative embodiments, detector 100 may be one part of a larger healthcare monitoring system that incorporates digital tracking of a user's progress, including, for example, dates of past scanning, results, scheduled future scanning, and the like. In embodiments, a web-based system may be integrated with the detector of the present invention. In embodiment, a smartphone-based system (e.g., iPhone, Android, Windows Phone) is integrated into the system for tracking or schedule purposes.

### CONCLUSION

It will be understood that there are numerous modifications of the illustrated embodiments described above which will be readily apparent to one skilled in the art, such as many variations and modifications of the compression connector assembly and/or its components including combinations of features disclosed herein that are individually disclosed or claimed herein, explicitly including additional combinations of such features, or alternatively other types of contact array connectors. Also, there are many possible variations in the materials and configurations. These modifications and/or combinations fall within the art to which this invention relates and are intended to be within the scope of the invention. It is noted, as is conventional, the use of a singular element in a claim is intended to cover one or more of such an element.

We claim:

1. A screening tool for use in detecting subcutaneous thermal abnormalities comprising:
an elongate handle portion;
a frame portion connected to said handle portion;
a thermochromatic screen supported by said frame portion;
wherein said thermochromatic screen is formed from an opaque backing material impregnated on at least one side by a plurality of thermal-sensitive liquid crystals; and
wherein said thermal-sensitive crystals are responsive to a predetermined temperature range for the desired application.

2. The screening tool of claim 1 wherein said elongate handle is formed from a material that is heat resistant such that the amount of heat transmitted from an operator's hand to said thermochromatic screen is minimized.

3. The screening tool of claim 1 wherein said elongate handle is formed from plastic, polyvinyl chloride, polypropylene, wood, ceramic, cardboard, or rubber.

4. The screening tool of claim 1 wherein said frame portion and said elongate handle portion are integrally formed from a single piece of material.

5. The screening tool of claim 1 wherein said frame portion and said elongate handle portion are permanently joined during manufacture.

6. The screening tool of claim 1 wherein said thermochromatic screen is joined to said frame portion such that said thermochromatic screen can be applied flush to a surface.

7. The screening tool of claim 1 wherein said frame portion comprises two discrete sections that are joined during manufacture and further wherein a portion of said thermochromatic screen is held between said discrete sections.

8. A screening tool for use in detecting subcutaneous thermal abnormalities comprising:
an plurality of opposing handle portions;
a thermochromatic screen formed from an opaque backing material impregnated on at least one side by a plurality of thermal-sensitive liquid crystals;
wherein at least two edges of said thermochromatic screen are substantially supported by said handle portions; and
wherein said thermal-sensitive crystals are responsive to a predetermined temperature range for the desired application.

9. The screening tool of claim 8 wherein said handle portions are formed from a material that is heat resistant such that the amount of heat transmitted from an operator's hand to said thermochromatic screen is minimized.

10. The screening tool of claim 8 wherein said handle portions are formed from plastic, polyvinyl chloride, polypropylene, wood, ceramic, cardboard, or rubber.

11. A method for detecting subcutaneous thermal abnormalities comprising:
providing a detector having a thermochromatic screen formed from an opaque backing material impregnated on at least one side by a plurality of thermal-sensitive liquid crystals, wherein said thermal-sensitive crystals are responsive to a predetermined temperature range for the desired application;
applying said detector to the surface of the region to be tested;
waiting for said thermochromatic screen to register; and
examining said thermochromatic screen for a evidence of a color gradient.
